(19) United States
(12) Patent Application Publication
(10) Pub. No.: US 2008/0032953 A1
(43) Pub. Date: Feb. 7, 2008

(54) Method For Inhibiting Prebiotic Effect Of Food Proteins

(75) Inventors: Tan Hung Nguyen, Saint Ave (FR); Renaud Sergheraert, Fabregues (FR)

Correspondence Address: IP GROUP OF DLA PIPER US LLP, ONE LIBERTY PLACE, 1650 MARKET ST, SUITE 4900, PHILADELPHIA, PA, 19103, US

(73) Assignee: Royal Canin SA,, Aimargues (FR)

(21) Appl. No.: 11/631,242

(22) PCT Filed: Jun. 16, 2005
(86) PCT No.: PCT/EP05/52804

§ 371 Date: Feb. 13, 2007

(30) Foreign Application Priority Data
Jun. 30, 2004 (FR) FR 0407216

(51) Int. Cl. A61K 31/66; A61P 43/00

(52) U.S. Cl. 514/106

(57) ABSTRACT

The invention concerns a method for inhibiting prebiotic effect of food proteins on the oral bacteria microflora of carnivorous domestic animals, said method consisting in administering to the carnivorous domestic animal an inhibitor of said prebiotic effect, said inhibitor comprising a water-soluble food phosphate. The inhibitor may be administered alone or via a food or a formulation veterinary or not.

The invention relates to a method for inhibiting the probiotic or prebiotic effect of food proteins with respect to the bacterial microflora of the oral cavity of carnivorous domestic animals. The invention consists in inhibiting this effect by using water-soluble food phosphates.

The oral cavity of dogs and cats harbors a varied bacterial microflora, which is classified into aerobic microflora and anaerobic microflora. This microflora is found on the oral mucous membranes, on the teeth and in the saliva, the latter, by virtue of its aqueous state, being the environment for the development and also the carrier for the diffusion of the microflora.

At birth, the animal's oral cavity is sterile, but it is rapidly colonized by aerobic and anaerobic bacteria as soon as the young animal absorbs food. Not only are these foods not sterile, but the proteins therein, which are diffused in the saliva, or have remained residual on the mucous membranes and on or between the teeth, promote the development of the microbial microflora. It is then said that these proteins have a prebiotic or probiotic effect with respect to the microflora (from pro, for, and bios, life, unlike antibiotic). It should be noted that saliva, which is produced sterilely by the salivary glands, could never have been an environment for the development and diffusion of the bacterial microflora without the presence of food proteins.

According to the present invention, the terms “prebiotic” or “probiotic” are used without distinction for defining the same effect of promoting the growth and/or the metabolic activity of microorganisms.

Unfortunately, in cats and dogs, oral hygiene is difficult to practice on a daily basis. Rinsing the mouth with disinfectants and scraping and brushing the teeth with toothpaste after meals, are not common practice as in humans. On the other hand, cats and dogs are increasingly well fed, often several times a day, either with “household” rations or with commercial foods that are called “petfoods”. The latter may be dry, moist or semi-moist foods, snacks or treats. Regardless of their origin or their presentation, all these foods provide proteins of animal or plant origin, which are admittedly necessary for the animals' nutrition, but which leave residues favorable to the development of the oral bacterial microflora.

The usual development of the microbial flora can lead to unwanted esthetic effects such as transient bad breath that needs to be controlled.

When this bacterial microflora develops excessively in the oral cavity, the host animal can exhibit numerous conditions well known to breeders and veterinarians, all the more so since cats and dogs have folds on their gums that form “gum pockets”:
halitosis (bad breath),
gingivitis (inflammation of the gum),
periodontitis or periodontal disease (inflammation of the periodont, i.e. of the assembly of tissues supporting and attaching the teeth),
pharyngitis (inflammation of the pharyngeal mucosa),
etc.

This excessive, pathological, development is associated with a problem in controlling the microbial flora which differs from the usual development that simply causes unwanted esthetic effects.

These conditions can be treated using antimicrobial agents (Trevor Chin Quee, Trianthi Roussou and E. C. S. Chan, “In vitro activity of Rodogyl against putative periodontopathic bacteria” Antimicrobial Agents and Chemotherapy, Vol. 24, No. 3, 1983, pp. 445-447; K. S. Kornman, B. Siegrist, W. A. Soskolne and K. Nuki, “The predominant cultivable subgingival flora of beagle dogs following ligature placement and metronidazole therapy”, Journal of Periodontal Research, Vol. 16, 1981, pp. 251-258).

However, these treatments with antimicrobial agents are often late, since they are only turned to when the conditions are already clearly visible. It is therefore essential to find means for decreasing the excessive development of the bacterial microflora of the oral cavity of cats and dogs, before it causes pathological conditions.

The Applicant has discovered, unexpectedly, that food phosphates are capable of inhibiting the prebiotic effect of food proteins on the microflora of the oral cavity of carnivorous domestic animals. It is essential for these phosphates to be water-soluble in order to be active in the animals' saliva. It is therefore preferable to use sodium pyrophosphates or polyphosphates. The phosphate can be taken in on its own, or through a food, or by means of any veterinary or nonveterinary preparation. In all cases, those skilled in the art will provide the phosphate to be used in an amount sufficient for it to be at least at a content of 0.50% of the saliva.

According to patent WO 93/25087 from the University of Indiana, phosphates, and particularly sodium hexametaphosphate, have already been used as sequestering and dissolving agents for preventing the formation of calcium crystals that constitute dental tartar in domestic animals. However, this prior art in no way described the inhibitory effect of phosphates on the prebiotic action of food proteins with regard to the bacterial microflora of the oral cavity.

The present invention therefore relates to a method for inhibiting the prebiotic effect of food proteins on the oral bacterial microflora of carnivorous domestic animals, said method consisting in administering to the carnivorous domestic animal a prebiotic effect inhibitor, the inhibitor comprising a water-soluble food phosphate.

This method of inhibition is a nontherapeutic method when it involves controlling the usual development of the bacterial flora.

This method of inhibition can be for therapeutic purposes when it involves controlling excessive development of the bacterial flora.

According to the present invention, the inhibitor may consist of a single water-soluble food phosphate or of a mixture of water-soluble food phosphates.

Water-soluble food phosphates are well known to those skilled in the art, particularly those authorized by Directive 70/524/EEC, published in the official journal of the European Union of 25 Feb. 2004.

Preferably, the water-soluble phosphate is different from a sodium hexametaphosphate, advantageously chosen from pyrophosphates and polyphosphates.

According to a preferred embodiment of the invention, the food phosphate is used in amounts such that it is dissolved at at least 0.5% in the animals' saliva.

According to the invention, the food phosphate can be administered on its own to the animals or can be provided as a mixture with foods for carnivorous domestic animals.

The foods are chosen from “household” rations, or dry, moist or semi-moist industrial foods, snacks or treats.

Advantageously, the amount of food phosphate in the supplemented food is greater than or equal to 1% by weight, preferably between and 2% by weight.

According to the invention, the inhibitor can be added to the foods extemporaneously, or else premixed.

According to another embodiment of the invention, the prebiotic effect inhibitor is administered to the carnivorous domestic animals in a veterinary or non-veterinary preparation.

The nonexhaustive and nonlimiting examples hereinafter make it possible to illustrate the invention.

### EXAMPLES

For all the experiments, the bacterial microflora of the oral cavity was removed, kept in a conservation medium, and then cultured in artificial saliva according to the following protocol:

Sampling of the Bacterial Microflora and Preparation of the Inoculum

Two male cats of “European” race, weighing approximately 5.50 kg, were anesthetized with 0.3 ml of a solution of medetomidine at 0.085 g/100 ml (Domitor, ND) and 0.26 ml of a solution of ketamine at 10 g/1000 ml (Imalgene 1000, ND).

In each animal thus immobilized, the saliva was sterilely suctioned with a pipette and the base of the teeth, the gums and the gum pockets were scraped with the back of a sterile scalpel.

All the samples were transferred and well-diluted in 100 ml of a sterile conservation medium (thiocolate resazurin medium from Biokar supplemented with 25% of glycerol). The conservation medium containing the samples was transferred into tubes with microbeads (Cryobilles, ND from AES). These tubes were then incubated for 6 hours in an incubator at 37° C., in a jar under CO₂, before being frozen for subsequent use.

When subsequently used, each tube is thawed at ambient temperature and then incubated for 12 hours at 37° C., in a jar under CO₂. The aerobic and anaerobic bacteria are counted according to the methods described hereinafter. The content of each tube is subsequently diluted with the sterile conservation medium so as to have an inoculum of 5000 (3.70 log10) revivable microorganisms in 0.2 ml.

Methods for Counting Bacteria

The aerobic microflora is cultured and then counted on a trypticase soy medium (Biokar) incubated at 37° C. for 48 hours.

The anaerobic microflora is cultured and then counted on a Schaedler medium (Biokar) supplemented with 5% of sterile defibrinated sheep blood, incubated at 37° C., in a jar under CO₂, for 48 hours.

Artificial Saliva

The basic artificial saliva is prepared according to table 31 on page 244 of the manual Biological Handbooks—Metabolism, compiled and edited by Philip L. Altman and Dorothy S. Dittmer, published by Federation of American Societies for Experimental Biology, 1968.

This basic saliva is supplemented with L-cysteine at a rate of 0.5 g/liter in order to lower its redox potential so as to be able to grow therein both the aerobic bacterial microflora and the anaerobic bacterial microflora. The whole will subsequently be referred to as “artificial saliva”.

Experimentation

The artificial saliva is dispensed into test tubes at a rate of 20 ml per tube. Each tube is supplemented or not supplemented with the protein, in the presence or absence of the phosphate to be tested. Each treatment is composed of two tubes.

The whole is autoclaved at 110° C. for 15 minutes.

Each tube is subsequently inoculated with 0.2 ml of inoculum described above. The whole is incubated in an incubator at 37° C., with shaking.

After 24, 48 or 72 hours of incubation, the aerobic flora and the anaerobic flora are counted according to the methods described above.

The result of each treatment is the mean of the counts of two test tubes, expressed as log10 of C.F.U. (Colony Forming Units) per ml of artificial saliva.

Experiment 1

The treatments were the incorporation of a dehydrated poultry meat meal (protein DSH, from the Société des Protéines Industrielles, 56230 Berric, France, with a titer of 70% of total nitrogenous matter) at a rate of, respectively, 0, 0.5, 1.0 and 1.5% into the artificial saliva.

Table 1 shows that, in the absence of the protein, the aerobic and anaerobic bacterial microflora do not grow, or grow with great difficulty, in the artificial saliva alone. However, once the protein is present, even at a content as low as 0.5%, both the aerobic microflora and the anaerobic microflora “rocket” from 24 hours of incubation.

This experiment clearly shows the prebiotic effect of this food protein on the oral bacterial microflora.

Experiment 2

In this experiment, the inhibition of the prebiotic effect of a dried hydrolysate of poultry protein (protein MP9007 from the Société des Protéines Industrielles having a titer of 72.5% of total nitrogenous matter), incorporated into the artificial saliva at a content of 1%, is tested in the presence of trisodium phosphate at contents of 5% or 10%.

Table 2 shows that the prebiotic effect of the MP9007 protein is completely inhibited by the trisodium phosphate incorporated at 10%, both with respect to the aerobic microflora and with respect to the anaerobic microflora.

The inhibitory effect of trisodium phosphate at 5%, although not total, is also very substantial from 24 hours of incubation.

Experiment 3

In this experiment, trisodium phosphate is tested again, but incorporated only at 0.5%, with respect to the prebiotic effect of the MP9007 protein incorporated into the artificial saliva at a content of 1%. Given the data from the previous experiment, the test is stopped after 24 hours of incubation.

Table 3 shows that the trisodium phosphate incorporated at 0.5% again decreases the prebiotic effect of the MP9007 protein, both with respect to the aerobic microflora (7.75. versus 8.16 log10) and the anaerobic microflora (7.75 versus 8.54 log10).

Experiment 4

The assay consists in testing the inhibitory effect of sodium tripolyphosphate incorporated at 0, 0.5, 1, 1.5 and 2%, on the prebiotic effect of the MP9007 protein incorporated at 1% into the artificial saliva.

Table 4 shows that sodium tripolyphosphate significantly inhibits the prebiotic effect of the protein.

Experiment 5

In this assay, the inhibitory effect of sodium tripolyphosphate incorporated at 0, 0.5, 1, 1.5 and 2% on the prebiotic effect of a dehydrated soya hydrolysate (Nurish 1500 IP, ND from Solea Company, having a titer of 83% of total nitrogenous matter) incorporated into the artificial saliva at 1%, is tested.

Table 5 shows that the sodium tripolyphosphate, regardless of the amount incorporated, virtually inhibits the entire prebiotic effect of the plant protein used.

Experiment 6

In this assay, the inhibitory effect of sodium tripolyphosphate incorporated at 0, 0.5, 1, 1.5 and 2%, on the prebiotic effect of a dehydrated soya hydrolysate (Nurish 1500 IP, ND from Solea Company, having a titer of 83% of total nitrogenous matter) incorporated into the artificial saliva at 0.5 and at 1%, was tested.

In this assay, oral microflora taken from a dog were used.

The results reported in table 6 show that sodium tripolyphosphate inhibits the prebiotic effect of the soya hydrolysate on canine aerobic and anaerobic oral microflora. The inhibitory effect is particularly substantial at tripolyphosphate incorporation rates greater than or equal to 1%.

TABLE 1
PREBIOTIC EFFECT OF THE DSH
PROTEIN ON THE ORAL MICROFLORA
(CFU/ml, mean in log10 of two tubes per treatment)

| Protein | | 0 | 24 | 48 | 72 |
|---|---|---|---|---|---|
| DSH | Microflora | hour | hours | hours | hours |
| 0% | Aerobic | 3.70 | <3.00 | <3.00 | 4.75 |
| | Anaerobic | 3.70 | <3.00 | <3.00 | <3.00 |
| 0.5% | Aerobic | 3.70 | 7.85 | 8.99 | 8.01 |
| | Anaerobic | 3.70 | 7.27 | 7.84 | 7.13 |
| 1.0% | Aerobic | 3.70 | 8.55 | 10.01 | 7.98 |
| | Anaerobic | 3.70 | 8.32 | 9.97 | 7.97 |
| 1.5% | Aerobic | 3.70 | 8.19 | 10.28 | 8.19 |
| | Anaerobic | 3.70 | 8.27 | 9.48 | 7.50 |

TABLE 2
INHIBITION OF THE PREBIOTIC EFFECT OF THE MP9007
PROTEIN BY TRISODIUM PYROPHOSPHATE AT 5 AND 10%
(CFU/ml, mean in log10 of two tubes per treatment)

| Protein | Trisodium | | 0 | 24 | 48 | 72 |
|---|---|---|---|---|---|---|
| MP9007 | pyrophosphate | Microflora | hour | hours | hours | hours |
| 0% | 0% | Aerobic | 3.70 | <4.00 | <4.00 | <4.00 |
| | | Anaerobic | 3.70 | <4.00 | <4.00 | <4.00 |
| 1% | 0% | Aerobic | 3.70 | 8.79 | 7.51 | 8.05 |
| | | Anaerobic | 3.70 | 8.43 | 7.72 | 7.59 |
| 1% | 5% | Aerobic | 3.70 | 6.88 | 7.56 | 7.69 |
| | | Anaerobic | 3.70 | 6.88 | 7.46 | 6.26 |
| 1% | 10% | Aerobic | 3.70 | <4.00 | 4.88 | <4.00 |
| | | Anaerobic | 3.70 | <4.00 | <4.00 | <4.00 |

TABLE 3
INHIBITION OF THE PREBIOTIC EFFECT OF THE MP9007
PROTEIN BY TRISODIUM PYROPHOSPHATE AT 0.5%
(CFU/ml, mean in log10 of two tubes per treatment)

| Protein | Trisodium | | 0 | 24 |
|---|---|---|---|---|
| MP9007 | pyrophosphate | Microflora | hour | hours |
| 0% | 0% | Aerobic | 3.70 | <3.00 |
| | | Anaerobic | 3.70 | <3.00 |
| 1% | 0% | Aerobic | 3.70 | 8.16 |
| | | Anaerobic | 3.70 | 8.54 |
| 1% | 0.5% | Aerobic | 3.70 | 7.75 |
| | | Anaerobic | 3.70 | 7.75 |

TABLE 4
INHIBITION OF THE PREBIOTIC EFFECT OF THE
MP9007 PROTEIN BY SODIUM TRIPOLYPHOSPHATE
(CFU/ml, mean in log10 of two tubes per treatment)

| Protein | Tripoly- | | 0 | 24 | 48 |
|---|---|---|---|---|---|
| MP9007 | phosphate | Microflora | hour | hours | hours |
| 1% | 0% | Aerobic | 3.70 | 7.97 | 7.69 |
| | | Anaerobic | 3.70 | 7.90 | 7.97 |
| 1% | 0.5% | Aerobic | 3.70 | 5.50 | 6.90 |
| | | Anaerobic | 3.70 | 5.41 | 7.04 |
| 1% | 1% | Aerobic | 3.70 | 4.81 | 6.49 |
| | | Anaerobic | 3.70 | <4.00 | 6.36 |
| 1% | 1.5% | Aerobic | 3.70 | 4.84 | 6.83 |
| | | Anaerobic | 3.70 | 4.98 | 6.82 |
| 1% | 2% | Aerobic | 3.70 | <4.00 | <4.00 |
| | | Anaerobic | 3.70 | <4.00 | <4.00 |

TABLE 5
INHIBITION OF THE PREBIOTIC EFFECT OF SOYA
HYDROLYSATE BY SODIUM TRIPOLYPHOSPHATE
(CFU/ml, mean in log10 of two tubes per treatment)

| Soya | Tripoly- | | 0 | 24 | 48 |
|---|---|---|---|---|---|
| hydrolysate | phosphate | Microflora | hour | hours | hours |
| 1% | 0% | Aerobic | 3.70 | 6.93 | 8.31 |
| | | Anaerobic | 3.70 | 7.11 | 8.10 |
| 1% | 0.5% | Aerobic | 3.70 | <4.00 | <4.00 |
| | | Anaerobic | 3.70 | <4.00 | <4.00 |
| 1% | 1% | Aerobic | 3.70 | <4.00 | <4.00 |
| | | Anaerobic | 3.70 | <4.00 | <4.00 |
| 1% | 1.5% | Aerobic | 3.70 | <4.00 | <4.00 |
| | | Anaerobic | 3.70 | <4.00 | <4.00 |
| 1% | 2% | Aerobic | 3.70 | <4.00 | <4.00 |
| | | Anaerobic | 3.70 | <4.00 | <4.00 |

TABLE 6
INHIBITION OF THE PREBIOTIC EFFECT OF THE
SOYA HYDROLYSATE WITH RESPECT TO A CANINE
ORAL FLORA BY SODIUM TRIPOLYPHOSPHATE
(CFU/ml, mean in log10 of two tubes per treatment)

| Tripoly- | Soya | | 0 | 24 | 48 |
|---|---|---|---|---|---|
| phosphate | hydrolysate | Microflora | hour | hours | hours |
| 0% | 0% | Aerobic | 3 | <3 | <3 |
| | | Anaerobic | 3 | <3 | <3 |
| 0% | 0.5% | Aerobic | 3 | 7.47 | 8.06 |
| | | Anaerobic | 3 | 6.67 | 6.94 |
| 0% | 1% | Aerobic | 3 | 7.94 | 8.01 |
| | | Anaerobic | 3 | 6.72 | 7.12 |
| 0.5% | 0.5% | Aerobic | 3 | 7.10 | 7.54 |
| | | Anaerobic | 3 | 1.92 | 5.80 |
| 0.5% | 1% | Aerobic | 3 | 7.50 | 7.51 |
| | | Anaerobic | 3 | 6.30 | 6.24 |
| 1% | 0.5% | Aerobic | 3 | <3 | <3 |
| | | Anaerobic | 3 | <3 | <3 |
| 1% | 1% | Aerobic | 3 | <3 | 3.17 |
| | | Anaerobic | 3 | <3 | 3.30 |
| 1.5% | 0.5% | Aerobic | 3 | <3 | <3 |
| | | Anaerobic | 3 | <3 | <3 |
| 1.5% | 1% | Aerobic | 3 | <3 | <3 |
| | | Anaerobic | 3 | <3 | <3 |
| 2% | 0.5% | Aerobic | 3 | <3 | <3 |
| | | Anaerobic | 3 | <3 | <3 |
| 2% | 1% | Aerobic | 3 | <3 | <3 |
| | | Anaerobic | 3 | <3 | <3 |

1. A method for inhibiting the prebiotic effect of food proteins on the oral bacterial microflora of carnivorous domestic animals, said method comprising administering to the carnivorous domestic animal a prebiotic effect inhibitor, wherein the inhibitor comprises a water-soluble food phosphate.

2. The method as claimed in claim 1, wherein the inhibitor is chosen from the group consisting of a sodium pyrophosphate and a sodium tripolyphosphate.

3. The method as claimed in claim 1, wherein the water-soluble food phosphate is dissolved at least 0.5% in the animals' saliva.

4. The method as claimed in claim 1, wherein the water-soluble food phosphate is administered on its own to the animals.

5. The method as claimed in claim 1, wherein the inhibitor is provided as a mixture with foods for carnivorous domestic animals.

6. The method as claimed in claim 5, wherein the inhibitor is added to the foods extemporaneously.

7. The method as claimed in claim 5, wherein the inhibitor is premixed with the foods.

8. The method as claimed in claim 5, wherein the foods are selected from the group consisting of household rations, dry industrial foods, moist industrial foods, semi-moist industrial foods, snacks and treats.

9. The method as claimed in claim 1, wherein the water-soluble food phosphate is administered to the animals through a veterinary preparation.

10. The method as claimed in claim 1, wherein the inhibitor comprises a mixture of water-soluble food phosphates.

11. (canceled)

12. The method as claimed in claim 1, wherein the water-soluble food phosphate is administered to the animals through a non-veterinary preparation.
